(19) United States
(12) Patent Application Publication
(10) Pub. No.: US 2008/0319418 A1
(43) Pub. Date: Dec. 25, 2008

(54) Catheter Steering Device

(75) Inventor: Evan K. Chong, South Strathfield (AU)

Correspondence Address: MORRISON & FOERSTER LLP, 755 PAGE MILL RD, PALO ALTO, CA, 94304-1018, US

(73) Assignee: CATHRX PTY LTD, Eveleigh, NSW (AU)

(21) Appl. No.: 11/547,433

(22) PCT Filed: Mar. 23, 2005
(86) PCT No.: PCT/AU2005/000421

§ 371 Date: Sep. 11, 2008

Related U.S. Application Data
(60) Provisional application No. 60/558,299, filed on Mar. 30, 2004.

(51) Int. Cl. A61M 25/01

(52) U.S. Cl. 604/528

(57) ABSTRACT

A catheter steering and shape forming device (10) comprises an clongate element (12) having a proximal end (14) and a distal end (16) and defining a longitudinal axis. A plurality of longitudinally spaced, transversely extending notches (20) is formed in the clongate element (12, the notches (20) having non-parallel sides and the notches (20) being arranged in at least one group (18) proximate the distal end (16) of the clongate element (12).

### CROSS-REFERENCE TO RELATED APPLICATIONS

The present application claims priority from U.S. Provisional Patent Application No. 60/558299 filed on 30 Mar. 2004, the contents of which are incorporated herein by reference.

### FIELD OF THE INVENTION

This invention relates to steering and shape forming devices for medical equipment. More particularly, the invention relates to a catheter steering and shape forming device.

### BACKGROUND TO THE INVENTION

Electrophysiology catheters are medical devices used for measuring electrical signals within the heart in the diagnosis of arrhythmias. Certain types of these catheters may also be used for treating arrhythmias through ablative techniques.

Generally, to access the region of the heart to be treated, the catheter is inserted through the femoral vein of the patient. The tip of the catheter therefore needs to be steered through the vascular system of the patient to the desired location. Similarly, the tip needs to be steered through the chambers of the heart to arrive at the desired location.

Steerable catheters have in the past made use of a metal strip or shim contained within the distal end of the catheter. One or more pull wires are connected to the metal strip. Manipulation of these pull wires causes the metal strip to bend and, in so doing, to deflect the distal end of the catheter.

This is a complex design which is difficult to manufacture. In particular, there are numerous components which are required to be joined together. These components must be assembled by hand.

In addition, a steering device for the catheter is, normally, contained within a lumen of the catheter. Most catheters have other elements in the lumen such as electrical conductors. Therefore, space within the lumen is at a premium.

### SUMMARY OF THE INVENTION

According to a first aspect of the invention, there is provided a catheter steering and shape forming device which comprises

an elongate element having a proximal end and a distal end and defining a longitudinal axis; and

a plurality of longitudinally spaced, transversely extending notches formed in the elongate element, the notches having non-parallel sides and the notches being arranged in at least one group proximate the distal end of the elongate element.

The device may include a plurality of groups of notches.

Preferably, the device includes one or more steering/shape forming control members with at least one control member being associated with each group of notches, the control members extending substantially parallel to the longitudinal axis of the elongate element. Each control member may be in the form of a pull wire. The pull wires may be attached at various positions relative to the groups of notches to create different shapes and directions of shape orientation. Each pull wire may be pulled independently or simultaneously with other pull wires to create different shapes and to move the distal end in different directions.

The elongate element may be tubular. Instead, the elongate element may be solid.

In a preferred embodiment, each notch may be substantially V-shaped in cross-section. This improves bendability of the elongate element resulting in smaller radii of curvature being able to be achieved. If desired, an apex of each notch may be shaped to provide stress relief. For example, the apex may have a bulbous formation or may be rounded to reduce sharp transitions where stress could occur in the elongate element.

In the case where the elongate element is tubular, the control members may be contained in a lumen of the elongate element. In the case where the elongate element is solid, one or more longitudinally extending grooves may be defined in a periphery of the elongate element. The, or each, groove may contain a control member.

As indicated above and to improve the flexibility of the steering device, the notches may be arranged in groups. A first group of notches, the first group having a first characteristic, may be arranged at a first part of the elongate member with a second group of notches, the second group having a second characteristic different from that of the first group, being arranged at a second, different part of the elongate element. The characteristic of each group may be a pitch, i.e. spacing between adjacent notches, or notches of a predetermined shape. By “shape” is meant the angle of the ‘V’ of each notch.

In one embodiment, the second part of the elongate element may be aligned with the first part along the longitudinal axis of the elongate element but may be circumferentially spaced from the first part of the elongate element. For example, the second group of notches may be offset 180° from the first group when viewing the elongate element along its longitudinal axis.

In another embodiment, the second part of the elongate element may be arranged proximally relative to the first part of the elongate element. The group of notches at the second part of the elongate element may be circumferentially spaced relative to the group of notches at the first part of the elongate element by less than 180°, preferably by about 90°. Thus, the second group of notches, containing one or two notches, may be arranged 90° out of alignment with the first group when viewing the elongate element along its longitudinal axis. With this arrangement, the first group of notches can be used to form the distal end of the elongate element into a predetermined shape, such as a loop. The second group of notches can then be used to position the loop in a plane transverse to the longitudinal axis of the elongate element, eg. at substantially 90° to the longitudinal axis of the elongate element.

According to a second aspect of the invention, there is provided a catheter steering and shape forming device which comprises an elongate element having a proximal end and a distal end and defining a longitudinal axis with material being removed in an interrupted manner in a longitudinal direction on one side of a medial plane passing through the longitudinal axis of the elongate element to facilitate steering and shape forming of the distal end of the elongate element.

According to a third aspect of the invention, there is provided a catheter which includes

a tubular member defining a lumen; and

a catheter steering and shape forming device, as described above, contained in the lumen of the tubular member.

The lumen of the tubular member may be unimpeded by other elements so that the device is a close fit in the lumen of the tubular member. By “close fit” means that there is very little, if any, lateral relative movement possible of the device relative to the lumen of the tubular member.

### BRIEF DESCRIPTION OF DRAWINGS

Embodiments of the invention are now described by way of example with reference to the accompanying drawings in which:

FIG. 1 shows schematic illustrations of a unidirectional catheter steering and shape forming device in an extended configuration and in a bent configuration;

FIG. 2 shows schematic illustrations of a bidirectional catheter steering and shape forming device in an extended configuration and in a bent configuration;

FIG. 3 shows a further embodiment of a catheter steering and shape forming device in an extended configuration;

FIG. 4 shows the device of FIG. 3 in an operative, bent configuration;

FIG. 5 shows schematic illustrations of three different forms of notches for a catheter steering and shape forming device;

FIG. 6 shows a three dimensional view of a further catheter steering and shape forming device; and

FIG. 7 shows a schematic, sectional end view of a catheter including the catheter steering and shape forming device.

### DETAILED DESCRIPTION OF EXEMPLARY EMBODIMENTS

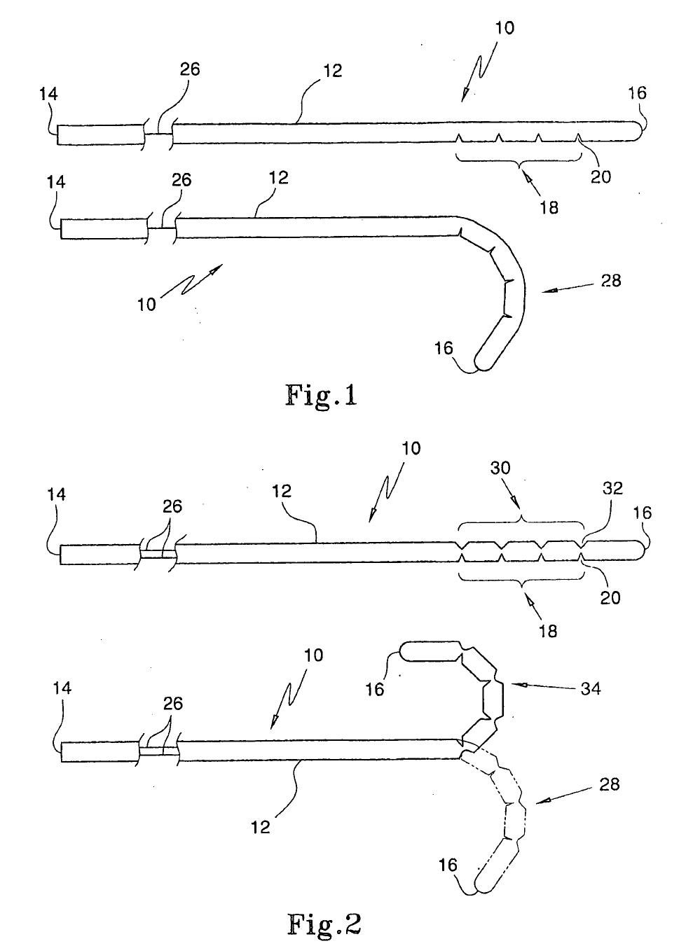

In FIG. 1 of the drawings, reference numeral 10 generally designates an exemplary embodiment of a catheter steering and shape forming device. For the sake of brevity, the catheter steering and shape forming device will be referred to below as the “steering device” or simply as the “device”. The steering device 10 comprise an elongate element 12 having a proximal end 14 and a distal end 16 and defining a longitudinal axis.

A bending facilitating arrangement, formed by the removal of material in ,n interrupted manner in a longitudinal direction, to form of a group 18 of notches 20 is arranged proximate the distal end 16 of the elongate element 12. Each notch 20 extends transversely to the longitudinal axis of the elongate element 12 and has non-parallel sides. In particular, each notch 20 is substantially V-shaped when viewed from an end of the notch 20.

As shown in FIG. 5 of the drawings, the notches 20 could, firstly, be non-symmetrical about a median plane, as shown by the steering device 10.1. In addition, or instead, to inhibit stress build-up in the elongate element 12, the apex of each notch 20 could be rounded for stress relief purposes as shown at 22 in respect of the steering element 10.2 in FIG. 5 of the drawings. Instead, each notch 20 could have a substantially keyhole-shaped cross-section having a bulbous apex 24 as shown with respect to the steering device 10.3 in FIG. 5 of the drawings.

The steering device 10 includes a steering/shape forming control member in the form of a pull wire 26. The pull wire 26 is attached to the distal end 16 of the elongate element 12. In the case of the embodiment of the invention shown in FIG. 1 of the drawings, because there is only one group 18 of notches 20, a single pull wire 26 is provided.

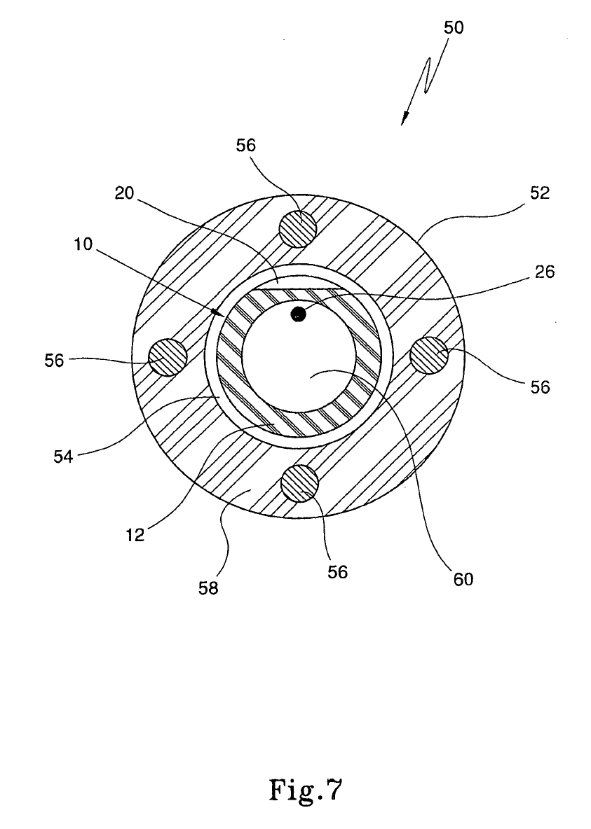

In use, the steering device 10 is inserted into a lumen 54 (FIG. 7) of a catheter 50 and, by manipulation of the pull wire 26, the tip of the catheter 50 is bent by bending the distal end 16 of the elongate element 12 as shown at 28 in FIG. 1 of the drawings. With the provision of the non-parallel sides of the notches 20, the bending of the distal end or tip 16 of the elongate element 12 is facilitated. In particular, smaller radii of curvature can be achieved than, for example, if the notches 20 had parallel sides. Hence, a much greater degree of manoeuvrability of the catheter containing the steering device 10 can be achieved.

Referring now to FIG. 2 of the drawings, a second embodiment of the steering device is illustrated. With reference to FIG. 1 of the drawings, like reference numerals refer to like parts, unless otherwise specified.

In this embodiment, in addition to the first group 18 of notches 20, the steering device includes a second group 30 of notches 32.

In this embodiment, the notches 32 have a wider V-shape than that of the notches 20. In other words, the walls of the notches 32 slope more gently than those of the notches 20. As a result, when it is desired to bend a catheter containing the steering device 10 about the notches 32 then, as shown at 34 in FIG. 2 of the drawings, because of the wider notches 32, a bend with a smaller radius of curvature than that shown at 28 can be achieved. This further improves the manoeuvrability of the steering device 10. Due to the two groups 18 and 30 of notches, two pull wires 26 are provided in the lumen of the elongate element 12, one for each group 18 and 30. These pull wires 26 are fixed either side of the neutral axis (which is coincident with the longitudinal axis of the elongate element 12).

Also, in this embodiment, a similar result can be achieved by having notches in groups where the pitch, i.e. the spacing between adjacent notches in one group, differs from the pitch in another group.

To provide the versatility of the steering device 10 shown in FIG. 2 of the drawings, the groups 18 and 30 of notches are longitudinally aligned but spaced circumferentially 180° from each other, when viewed from an end of the elongate element 12. Other groups (not shown) of notches could be provided 90° out of alignment with the groups 18 and 30.

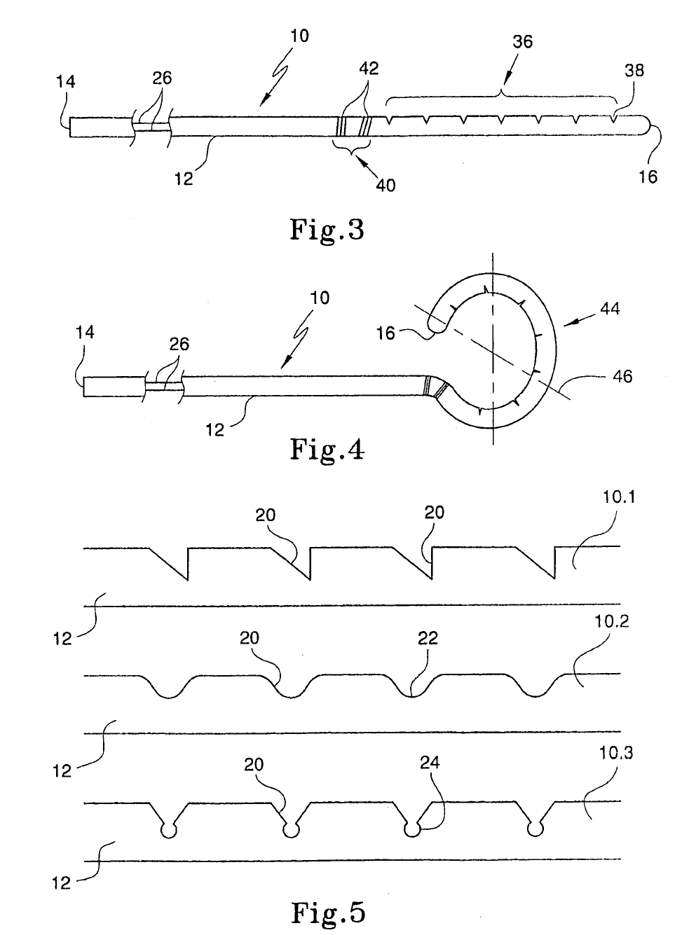

In the embodiment shown in FIG. 3 of the drawings, a first group 36 of notches 38 is provided proximate the distal end 16 of the elongate element 12. A second group 40 of notches 42 is proximally arranged relative to the first roup 36. The second group 40 comprises two notches 42. The second group 40 is spaced circumferentially by about 90° relative to the group 36 of notches 38.

The group 36 of notches 38 is used to form a loop 44 (FIG. 4). The elongate element 12 is then manipulated about the second group 40 of notches 42 via the associated pull wire 26 so that the loop 44 is arranged in a plane 46 transverse to the longitudinal axis of the elongate element 12. This facilitates use of the catheter for sensing and/or ablative purposes in a blood vessel, such as a pulmonary vein, or at an ostium to the blood vessel.

It will be appreciated that, by appropriate selection of notch shapes and pitch, any shape of loop, not necessarily circular, can be achieved. Thus, for example, spirals, any polygonal shape, S-shapes or arcs and shapes in multiple planes can be achieved.

In the embodiments illustrated in FIGS. 1 to 4 of the drawings, the elongate element 12 is a tubular member and the control members 26 are contained within a lumen 60 (FIG. 7) of the elongate element 12.

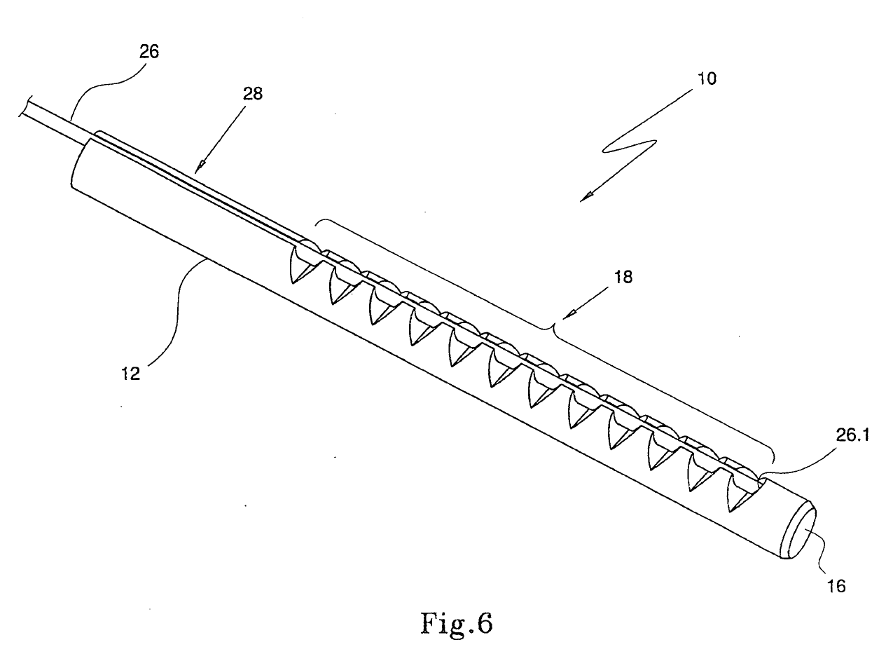

In the embodiment shown in FIG. 6 of the drawings, the elongate element 12 is solid. Once again, with reference to the previous drawings, like reference numerals refer to like parts, unless otherwise specified.

Because the elongate element 12 is solid, there is no lumen through which the pull wires 26 can extend. Instead, a longitudinally extending groove 28 is defined in the periphery of the elongate element 12. In this embodiment only a single group 18 of notches 20 is provided. It will be appreciated that, where there is more than one group of notches, a control member 26 is provided for each group of notches, each steering member 26 being located in its own longitudinally extending groove 28.

The control member 26 is anchored at 26.1 at the distal end 16 of the elongate element 12 but is otherwise free to slide in the groove 28 to facilitate bending of the elongate element 12 for use.

The control member 26 is held captive in the groove 28 either by means of a sleeve (not shown) or, when the steering device 10 is inserted into a lumen of the catheter, the periphery of the elongate element 12 abuts an interior surface of a wall of the lumen in a close fit so that the, or each, control member 26 is held captive in its associated groove 28. In this regard, the steering device 10 is intended particularly for use with the Applicant's catheter manufactured in accordance with the method described in its corresponding International Patent Application No. PCT/AU01/01339 dated 19 Oct. 2001 and entitled “An electrical lead”. The contents of that application are incorporated in this specification by reference.

Thus, with reference to FIG. 7 of the drawings, a catheter 50 is shown. The catheter 50 comprises an elongate tubular member 52 defining a lumen 54. Electrical conductors 56 are embedded in a wall 58 of the tubular member 52 so that the lumen 54 is, effectively, unimpeded by conductors and other elements which may occlude the lumen 54. Thus, the steering device 10, upon being received in the lumen 54 of the catheter 50 is a close fit in the lumen 54. In particular, in the case of the embodiment shown in FIG. 6 of the drawings, the internal surface of the wall 58 of the tubular member 52 aids in holding the control member 26 captive in its associated groove 28.

It is to be noted that the steering device 10 shown in FIG. 7 of the drawings is one in which the elongate element 12 is tubular, not solid.

The elongate element 12 of the steering device 10 may be of any suitable material having sufficient strength to withstand the induced compressive load when the pull wires 26 are manipulated. Materials include, but are not limited to, polymers, including reinforced polymers (e.g., with a metal wire braid or coil) and metal tubing. Additionally, composite materials such as a polymer tube with a central flat metal ribbon core could be used.

Depending on the application of the steering device 10, an outer jacket (not shown) may be used to cover the elongate element 12 to inhibit ingress of fluids.

The notches (and grooves where applicable) could be formed in the elongate element 12 by cutting, such as laser cutting or electric discharge machining (EDM). Instead, the notches and, where applicable, the grooves, could be formed by chemical means such as photochemical machining or etching. In the latter case, a work piece to be machined is thoroughly cleaned prior to being coated with a photo resist. The work piece is placed in a pre-formed double-sided photo tool which is used to selectively expose a photographic form according to CAD data.

When the photo resist-coated work piece is placed in the photo tool and exposed to ultraviolet light, an image is formed in the photo resist.

This is subsequently developed in a liquid formulation to form a durable image on both sides of the work piece. The work piece is immersed in an aqueous solution of ferric chloride for stainless steel (or an appropriate etchant when Nitinol is used) which selectively etches regions not protected by the photo resist to form the notches and, where applicable, the grooves. Finally, the resist is stripped and the metal surface of the work piece is cleaned.

It is an advantage of the invention, that a catheter steering and shape forming device 10 is provided which enables accurate control of deflection and shape forming of an end of a catheter to be achieved without the need for complicated steering components. By adjusting sizes of the notches and pitch, large variations in deflection and shapes can be obtained, even in multiple planes. In addition, the steering device 10 is a close fit in the lumen of a catheter obviating the need for any external sleeves and additional covering components and also allowing for finer control of the distal end of the catheter. The cost of the device 10 is therefore reduced as is the time taken to assemble the device 10. This once again reduces the cost of the device 10.

Still further, the modular nature of the catheter 50 facilitates replacement of one configuration of steering device 10 with another configuration of steering device to enable different radii of curvature and/or shapes to be achieved. This further improves the versatility of the catheter 50.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1: A catheter steering and shape forming device which comprises
an elongate element having a proximal end and a distal end and defining a longitudinal axis; and
a plurality of longitudinally spaced, transversely extending notches formed in the elongate element, the notches having non-parallel sides and the notches being arranged in at least one group proximate the distal end of the elongate element, the notches being arranged in at least two longitudinally spaced groups a first group of notches arranged at a first part of the elongate element facilitating the formation of the first part of the elongate element into a predetermined shape and a second group of notches located at a second part of the elongate element proximally arranged relative to the first part of the elongate element being circumferentially offset with respect to the first group of notches to facilitate movement of the loop into a plane transverse to the longitudinal axis of the elongate element.

2. (canceled)

3: The device of claim 1 which includes one or more steering/shape forming control members with at least one control member being associated with each group of notches, the control members extending substantially parallel to the longitudinal axis of the elongate element.

4: The device of claim 1 in which the elongate element is tubular.

5: The device of claim 1 in which the elongate element is solid.

6: The device of claim 1 in which each notch is substantially V-shaped in cross-section.

7: The device of claim 6 in which an apex of each notch is shaped to provide stress relief.

8-10. (canceled)

11: The device of claim 1 in which the group of notches at the second part of the elongate element is circumferentially spaced relative to the group of notches at the first part of the elongate element by less than 180°.

12. (canceled)

13: A catheter which includes
a tubular member defining a lumen; and
a catheter steering and shape forming device, as claimed in claim 1, contained in the lumen of the tubular member.

14: The catheter of claim 13 in which the lumen of the tubular member is unimpeded by other elements so that the device is a close fit in the lumen of the tubular member.
